(19) United States
(12) Patent Application Publication
(10) Pub. No.: US 2026/0273303 A1
(43) Pub. Date: Sep. 17, 2026

(54) SYSTEMS AND METHODS FOR TISSUE TREATMENT WITH AN ASYMMETRIC BEAM

(71) Applicant: Avava, Inc., Waltham, MA (US)

(72) Inventors: Rajender Katkam, Boston, MA (US); Jayant Bhawalkar, Auburndale, MA (US)

(21) Appl. No.: 19/473,472

(22) PCT Filed: Apr. 10, 2024
(86) PCT No.: PCT/US2024/023896

§ 371 Date: Oct. 7, 2025

Related U.S. Application Data
(60) Provisional application No. 63/458,597, filed on Apr. 11, 2023.

(51) Int. Cl. A61N 5/06; A61N 5/067

(52) U.S. Cl. CPC A61N 5/0616; A61N 5/067; A61N 2005/066

(57) ABSTRACT

Systems and method for tissue treatment are provided. The systems include an electromagnetic radiation (EMR) source configured to generate a treatment EMR beam, a plurality of optical elements configured to shape and focus the treatment EMR beam to a focal point within a tissue, a window assembly located down-beam from the plurality of optical elements and configured to contact the tissue, and a controller configured to control the EMR source to generate the treatment EMR beam during a treatment procedure. The first axicon and the second axicon are configured to shape the treatment EMR beam such that the shaped EMR beam has a transverse asymmetric energy profile. The plurality of optical elements include a first axicon and a second axicon, and the window assembly is configured to transmit the treatment EMR beam.

### CROSS-REFERENCE TO RELATED APPLICATIONS

This application claims the benefit of U.S. Provisional Application No. 63/458,597, filed Apr. 11, 2023, and entitled “Systems and Methods for Tissue Treatment with an Asymmetric Beam.” The entire contents of which is hereby incorporated herein by reference in its entirety.

### BACKGROUND

Lasers and light-based therapeutic devices are widely used in various medical fields, including in the field of dermatology, to deliver optical energy to target tissue. This delivery of optical energy can alter tissue, or chromophores present in tissue, through several mechanisms, including photothermal and photochemical mechanisms, and these alterations have been harnessed for well-known laser/light-based procedures, including laser hair removal, tattoo removal, removal of pigmented lesions and vascular lesions, tissue tightening, reduction in wrinkles, etc. Such devices in dermatology, for example, typically include a handpiece which is manually moved across a patient's skin by a physician. These devices deposit optical energy in short bursts to create fractional patterns comprising numerous microthermal zones (MTZ) of tissue damage that leave the majority of the skin tissue intact and untreated in order to improve a healing response of the treated tissue regions. The delivery of too little optical energy to the treatment area can result in an ineffective treatment, while the delivery of too much optical energy can result in undesirable tissue damage, e.g., burns. Further, the delivery of energy while the handpiece is moved across the patient's skin can result in the creation of fractional patterns that are less than ideal, such as fractional patents that appear smeared or blurred when compared to an ideal fractional pattern, resulting in both the reduction of energy density and increase in size of each MTZ, ultimately leading to a decreased treatment efficacy. It is therefore desirable that a treatment system and method be made available that addresses these limitations and challenges.

### SUMMARY

Systems and method for treatment of tissue are provided.

In an embodiment, a system is provided that can include an electromagnetic (EMR) source configured to generate a treatment EMR beam, a plurality of optical elements configured to shape and focus the treatment EMR beam to a focal point within a tissue, a window assembly located down-beam from the plurality of optical elements and configured to contact the tissue, and a controller configured to control the EMR source to generate the treatment EMR beam during a treatment procedure. The first axicon and the second axicon can be configured to shape the treatment EMR beam such that the shaped EMR beam has a transverse asymmetric energy profile. The plurality of optical elements can include a first axicon and a second axicon, and the window assembly can be configured to transmit the treatment EMR beam.

The system can vary in a number of ways. For example, in some variations, the transverse asymmetric energy profile can be at least partially annular. In some aspects, the transverse asymmetric energy profile can be a crescent. For example, the transverse asymmetric energy profile can be a substantially elongated ring. For example, the first axicon and the second axicon can be misaligned relative to each other. In some aspects, the first axicon can be translated relative to the second axicon. In other aspects, the first axicon can be rotated relative to the second axicon.

In another embodiment, a method is provided, including contacting a tissue with a window assembly of a treatment device, generating a treatment electromagnetic radiation (EMR) beam with an EMR source in electronic communication with the treatment device, and shaping and focusing the treatment EMR beam toward the tissue with a plurality of optical elements to create one or more treatment zones on the tissue. The plurality of optical elements can shape the beam such that the shaped EMR beam has a transverse asymmetric energy profile.

The method can vary in a number of ways. For example, the plurality of optical elements can include a first axicon and a second axicon being misaligned relative to an optical axis therethrough. In some aspects, the first axicon can be translated relative to the second axicon. In other aspects, the first axicon can be rotated relative to the second axicon. In still further aspects, the method can include collimating the treatment EMR beam with a collimating lens. In such further aspects, the first axicon and second axicon can be misaligned relative to the collimating lens. For example, the one or more treatment zones can be at least partially annular. In some aspects, the one or more treatment zones can be crescent-shaped. For example, the one or more treatment zones can be a substantially elliptical ring. In some aspects, the one or more treatment zones can be substantially elliptical rings having a major axis aligned with a direction of tightening. For example, the method can include pulsing the treatment EMR beam while moving the treatment device relative to the tissue to create the one or more treatment zones.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the disclosure will be more fully understood from the following detailed description taken in conjunction with the accompanying drawings, in which:

FIG. 1A is diagram of fluence levels of a low-NA beam as related to depth of the beam within a tissue;

FIG. 1B is a diagram of fluence levels of a high-NA beam as related to depth of the beam within a tissue;

FIG. 2 is an image of tissue treated with a high-NA beam;

FIG. 3A is a cross-sectional image of a tissue slice treated with a high-NA beam at 10 mJ;

FIG. 3B is a cross-sectional image of a tissue slice treated with a high-NA beam at 20 mJ;

FIG. 3C is a cross-sectional image of a tissue slice treated with a high-NA beam at 40 mJ;

FIG. 3D is a cross-sectional image of a tissue slice treated with a high-NA beam at 70 mJ;

FIG. 3E is a cross-sectional image of a tissue slice treated with a high-NA beam at 200 mJ;

FIG. 4A is a perspective view of a treatment system according to an embodiment;

FIG. 4B is a cross-sectional view of a treatment device from the treatment system of FIG. 4A according to an embodiment;

FIG. 5A is a schematic view of a collimating and beam-shaping optical arrangement usable with the treatment device of FIG. 1;

FIG. 5B is a schematic view of the collimaging and beam-shaping optical arrangement of FIG. 5A according to a variation;

FIG. 5C is a schematic view of the collimating and beam-shaping optical arrangement of FIG. 5A according to another variation;

FIG. 6 is a top view of a micro-thermal zone (MTZ) created by a standard treatment beam;

FIG. 7A is a top view of an MTZ created by a treatment beam having a transverse energy profile, according to an embodiment;

FIG. 7B is a plot of an energy distribution delivered by the treatment beam of FIG. 7A;

FIG. 8A is a top view of an MTZ created by a treatment beam having a transverse asymmetric energy profile, according to an embodiment;

FIG. 8B is a plot of an energy distribution delivered by the treatment beam of FIG. 8A, according to an example;

FIG. 8C is a plot of an energy distribution delivered by the treatment beam of FIG. 8A, according to another example;

FIG. 9A is a photograph of a skin tissue treated by an asymmetric beam and displaying MENDs having a crescent-like shape;

FIG. 9B is another photograph of a skin tissue treated by an asymmetric beam and displaying MENDs having a crescent-like shape;

FIG. 9C is still another photograph of a skin tissue treated by an asymmetric beam and displaying MENDs having a crescent-like shape;

FIG. 10A is a photograph of a histology of a dyed skin tissue treated by an asymmetric beam and displaying lesions having a crescent-like shape;

FIG. 10B is a photograph of a histology of a cross-section of a dyed skin tissue treated by an asymmetric beam;

FIG. 10C is another photograph of a histology of a cross-section of a dyed skin tissue treated by an asymmetric beam;

FIG. 10D is another photograph of a histology of a cross-section of a dyed skin tissue treated by an asymmetric beam;

FIG. 10E is another photograph of a histology of a cross-section of a dyed skin tissue treated by an asymmetric beam;

FIG. 11A is an array of MTZs created by a beam having a transverse energy profile according to an embodiment; and

FIG. 11B is an array of MTZs created by a beam having an asymmetric energy profile according to an embodiment.

It is noted that the drawings are not necessarily to scale. The drawings are intended to depict only typical aspects of the subject matter disclosed herein, and therefore should not be considered as limiting the scope of the disclosure. The systems, devices, and methods specifically described herein and illustrated in the accompanying drawings are non-limiting exemplary embodiments.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

Embodiments of the disclosure are discussed in detail below with respect to fractionated treatment including skin rejuvenation and skin resurfacing, for example skin resurfacing for: acne, chickenpox and surgical scars, periorbital and perioral wrinkles, photoageing changes, facial dyschromias, and stretch marks. Additional treatments related to the disclosure include treatment of pigmentary conditions of the skin, such as melasma, and other pigmentary conditions, such as granuloma annulare.

The disclosed embodiments can be employed for treatment of other pigmentary and non-pigmentary conditions and other tissue and non-tissue targets without limit. Examples of pigmentary conditions can include, but are not limited to, post inflammatory hyperpigmentation (PIH), dark skin surrounding eyes, dark eyes, café au lait patches, Becker's nevi, Nevus of Ota, congenital melanocytic nevi, ephelides (freckles) and lentigo. Additional examples of pigmented tissues and structures that can be treated include, but are not limited to, hemosiderin rich structures, pigmented gallstones, tattoo-containing tissues, and lutein, zeaxanthin, rhodopsin, carotenoid, biliverdin, bilirubin and hemoglobin rich structures. Examples of targets for the treatment of non-pigmented structures, tissues and conditions can include, but are not limited to, hair follicles, hair shafts, vascular lesions, infectious conditions, sebaceous glands, acne, and the like.

Methods of treating various skin conditions, such as for cosmetic purposes, can be carried out using the systems described herein. It is understood that, although such methods can be conducted by a physician, non-physicians, such as aestheticians and other suitably trained personnel may use the systems described herein to treat various skin conditions with and without the supervision of a physician.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

Certain treatment procedures relying on fractionated treatment can make use of a variety of beam shapes in order to more effectively treat the above-described maladies. During such treatment procedures, a laser beam of optical energy is directed toward a target tissue, such as skin, in order to introduce a desired dose of optical energy to that target tissue to treat one or more ailments, such as those described above. The optical energy creates treatment areas known as micro-thermal zones (MTZs) of damage in a pattern or array. The delivery of too much optical energy can cause harm to the target tissue, while the delivery of too little optical energy can lead to an ineffective treatment. Further, it is known that creating MTZs having various shapes can provide certain benefits, including increasing an effectiveness of the treatment procedure when compared to treatments involving MTZs of different shapes, even when a total amount of energy is held constant. For example, the use of a focused transverse annular (ring-like) laser beam for treatment of skin can be more effective than a solid laser beam of otherwise-equivalent properties. With a beam having a transverse annular energy profile treats skin tissue, MTZs having an annular or ring-like shape can be created on the target tissue. The benefit of using an annular ring to create annular MTZs when compared to a solid beam is that the undamaged tissue at the center of the annular MTZs promotes healing of the coagulated tissue from the inside outward, as well as the outside inward. In contrast, a solid circular MTZ resulting from a solid beam only allows for healing from the outside inward. This promotion of healing can decrease adverse side effects resulting from a treatment with an annular beam, and additional optical energy can potentially be delivered to an afflicted tissue area without resulting in harm that would otherwise befall a treatment procedure using a solid beam.

A beam having a transverse annular energy profile can be created in a number of ways, including by using a pair of axicons. Laser treatment devices employing a pair of axicons and similar techniques to create such an annular beam, as well as methods associated with such devices, are disclosed in U.S. Patent Publication No. 2021/0138261, entitled “Feedback Detection for a Treatment Device,” which is hereby incorporated by reference herein in its entirety.

More specifically, improved treatment results can be achieved using new laser systems employing a highly focused laser beam that is capable of precise depth control in combination with simultaneous imaging and rapid scanning over large skin areas without being limited to a particular wavelength of laser light. Such systems are safe and effective, even among patients having darkly-pigmented skin. With conventional laser treatment devices, a large-area beam is delivered with low numerical aperture (NA), resulting in a nearly-collimated beam, and the delivered energy is varied to adjust fluence for treatment. NA is related to the apex angle of the cone of the focused laser light, and it is defined as the tangent of the half-angle of the cone such that the higher the NA of the system, the large the angle of the cone of energy delivered. Fluence is defined as energy per unit area.

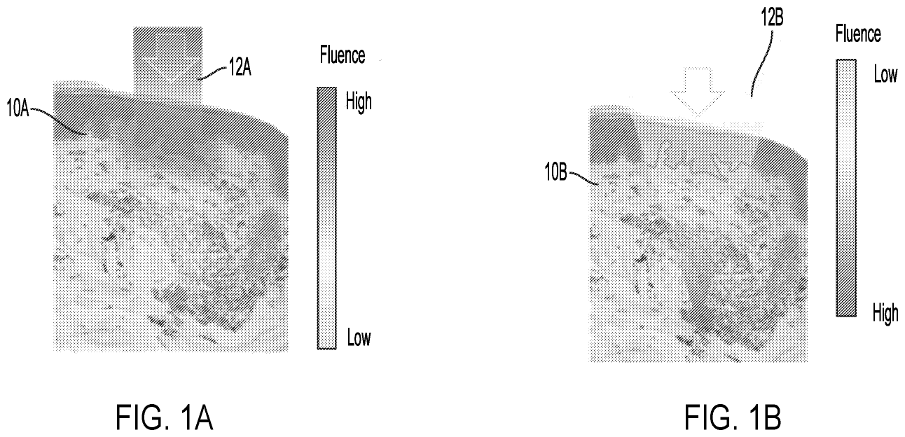

Optical scattering and absorption can result in a decrease in fluence as depth increases in these conventional treatment systems, but in a system with high NA, fluence rapidly increases with depth until the focal point of the beam is reached because the cross-sectional area of the conical beam decreases with depth to the focal point. This beam shape results in a large increase in fluence despite optical scattering and absorption, thus focusing the beam at depth with a high-NA optical system can result in reduced fluence at or near the tissue surface while producing high fluence at the focal point of the beam at a certain depth in the tissue. For example, in skin tissue, reduced fluence can be experienced by the epidermis, while high fluence can be experienced by the dermis. Fluence at the focal spot can be, for example, about a million times higher than fluence at a surface of the tissue. This effect can be seen in FIGS. 1A and 1, which respectively depict fluence delivered to tissue 10A, 10B using a low-NA (essentially collimated) beam 12A and a high-NA (conical) beam 12B.

Ultimately, the depth to which a high-NA beam is focused is arbitrary, being limited by absorption and scattering of the selected wavelength in the tissue. As the focal point of the beam moves deeper into the tissue, the fluence at the tissue surface decreases by a factor equal to the inverse square of the depth of the focal point. It is therefore possible to deliver therapeutic energy to a known depth and location within tissue while limiting surface fluence to safe levels. For example, when applied to skin tissue, energy can be focused to a location within the dermis while limiting epidermal fluence, even in patients with a highly pigmented dermis. For wavelengths of light that are strongly absorbed by water, such as in non-ablative fractional treatments, a focused beam serves to compensate for energy loss due to absorption. For wavelengths that not absorbed very well by water, a large fluence and/or power density at the focus can be achieved at the focus to thereby activate selective photothermolysis with exceptional depth control, as well as alternate non-thermal mechanisms of action including cavitation for targeted tissue cutting, laser-induced optical breakdown (LIOB), photochemical and/or mechanical modes of interaction.

Using software control and a scanning system, these described systems can achieve essentially any pattern of micro-scale damage within tissue, which can be further enhanced with the addition of onboard imaging capabilities. Onboard imaging can allow the detection of microscopic targets in the skin. For example, with such a system, it can be possible to download a new treatment capability without any change in hardware, and it can also be possible to acquire targets that are not specifically pigmented yet can be imaged, such as sebaceous glands, white hair, sensory nerves, etc.

In the studies described below, the laser treatment system used was based on a 1550 nm, erbium-fiber laser. A galvanometric mirror-based scanning system provided high speed x-y scanning of the focused ring microbeam in addition to depth control of the focused laser beam. The focused laser beam was transmitted through a cold sapphire window in contact with skin during treatment. Additionally, the sapphire window in the laser handpiece was actively cooled to a temperature that could be manually adjusted down to −10° C. for additional epidermal protection as well as patient comfort. A custom refractive index-matching gel was used to achieve optical coupling of the sapphire window to the skin and to also help the handpiece to glide easily over the treatment area. The laser and scanning system were synchronized to deliver an array of focused microbeams to the skin at the desired density and pulse energy selected by the user. The laser was operated at a fixed optical power and the pulse duration was varied to achieve the desired microbeam energy.

The treatment system also included the capability for wide-field imaging of the skin. Imaging was performed through the same optical path used for treatment, providing the advantage that the area being targeted by the laser falls directly in the center of the field of view of the imaging system. The imaging system provided a magnification of 1:1, and the images were captured by a Sony IMX252 camera with a pixel size of 3.5 micrometers. As a result, the imaging system could achieve better than 10 micrometer resolution of the skin surface and capture images at a frame rate of 60 frames per second. Illumination of the skin was achieved by LEDs located on the perimeter of the treatment tip of the device, which ensures consistency between images regardless of ambient lighting.

Ex vivo experiments were performed using human abdominal skin samples donated by patients undergoing abdominoplasty surgery. The skin was treated with the above-described laser system operating at 1550 nm in order to determine the physical dimensions of individual MTZs with varying pulse energies and focus depths. Prior to laser treatment, the subcutaneous fat was removed, sparing 2 mm of fat on the sample. The skin sample was then placed on saline-soaked gauze pads and heated with a digital dry bath until the skin surface reached a temperature of 32° C.±2° C. as measured with an infrared thermometer. Each skin treatment area was marked, and hairs were gently shaved before laser treatment as needed using a razor blade. Immediately prior to treatment, the skin was sanitized with 70% isopropyl alcohol and gauze, and a small drop of the coupling gel was placed for refractive index-matching between the sapphire window and the skin and also to reduce friction for ease of moving the handpiece over the skin.

Pulse energies for the treatments were carried out at 20 mJ, 40 mJ, 70 mJ, 100 mJ, 150 mJ, and 200 mJ, each of which were focused at multiple levels from the surface to the dermis for each given pulse energy. The spot densities were titrated between 81 MTZ/cm² (1200 micrometer spot pitch) and 289 MTZ/cm² (600 micrometer spot pitch) to ensure no overlap of MTZs. A single pass of treatment was performed. Samples were then frozen at −20° C. and cut into smaller specimens (approximately 10 mm×10 mm each) representing a single treatment parameter, embedded in an optimal cutting temperature compound, and then sectioned using Leica cryostat. The samples were sliced into 20 micrometer-thick sections and stained with nitro blue tetrazolium chloride (NBTC) and H&E stains collaterally. The stained samples were imaged at 20× using a digital slide scanner. Slides were analyzed by a dermatopathologist.

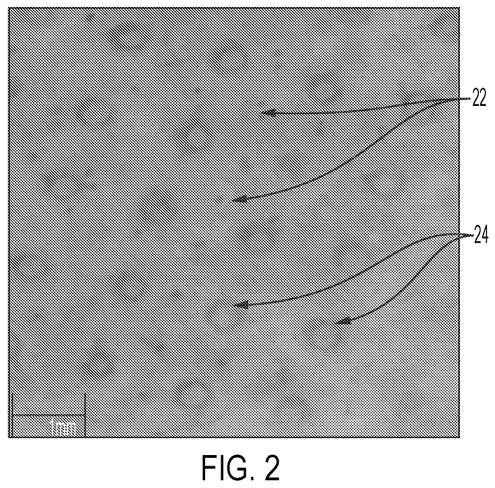

FIG. 2 shows a high-resolution image of treated tissue collected by the integrated wide-field imaging system. Superficial focus and deep focus resulting in MTZs, respectively shown as dots 22 and rings 24, can be seen in FIG. 2, which are further depicted between one and three days post-treatment. The patterns (shape, depth) of thermal injury were similar after ex vivo and in vivo exposures, and varied predictably with the energy delivered per microbeam, and with focus depth.

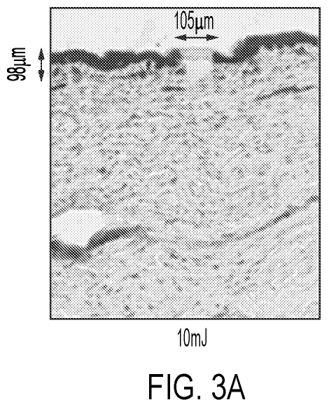

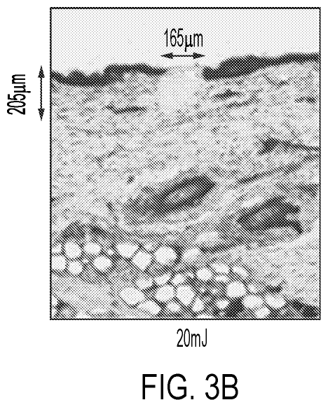

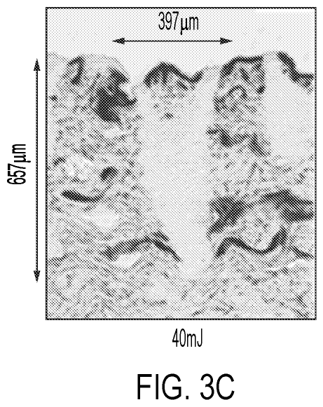

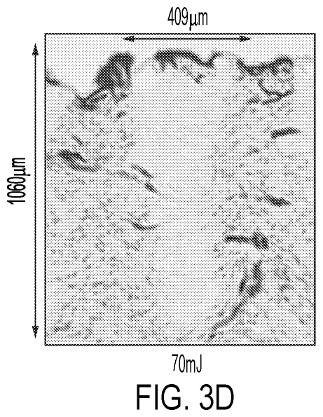

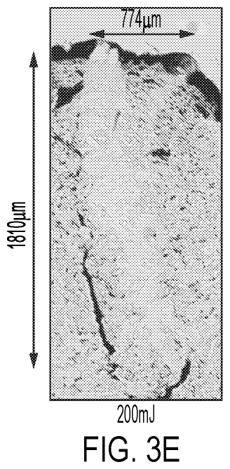

Namely, lesion depth increases with increasing focus depth, and so does the energy required to create the lesion due to the increasing volume of coagulation with increasing depth. FIGS. 3A-3E show the lesion profile at increasing depths with correspondingly increasing energy of 10, 20, 40, 70, and 200 mJ. Two distinct patterns of thermal injury were observed. When focused at low pulse energy (10-20 mJ) at or near the skin surface (0-100 μm of depth), a microscale localized region of epidermal injury was produced, with or without extent into the underlying dermis depending on the energy. Although 1550 nm is generally believed to cause coagulation to depths of several hundred micrometers, a superficial focus using high NA optics can confine the lesion depth to under 200 μm, similar to what is obtained with a much higher water-absorbed wavelength such as 1927 nm. This demonstrates the ability of these systems to selectively tune the lesion depth without the need for changing the wavelength. Furthermore, the diameter of injury zones when focused at the skin surface was only about 100 μm, substantially less than the ~200-300 μm lesions of current non-ablative fractional laser treatments. The smaller diameter of injury zone is a consequence of high-NA focusing of the single-mode erbium fiber laser beam. Reducing the diameter of epidermal injury zone 2-3-fold amounts to a 4-9-fold reduction in the amount of epidermis injured per microbeam exposure.

At higher energy levels, i.e., greater than 40 mJ, and deeper focus depths the coagulation pattern was a cone matching the numerical aperture of the conical beam, with a central zone of epidermal sparing. The extent of the zone of central sparing was found to decrease for higher energies and shallower focus depths. FIGS. 3A-3E demonstrate the ability of these systems to generate precise lesion geometries from superficial (sub-100 microns) to as deep as 1800 μm using a single wavelength.

Ultimately, investigation of tissue interaction and clinical effects of the use of a 1550 nm wavelength conical-beam, high-numerical-aperture prototype system yields promising results. Such systems are feasible and well tolerated for up to 200 mJ microbeam energy (well above levels associated with conventional 1550 nm fractional laser treatment) with no major adverse events with the potential to achieve quantifiable results in patients with various skin conditions. The clinical impact of a practical focused laser treatment system is broad.

The advantages realized by the above-described systems utilizing a beam having a transverse annular energy profile are significant, especially when compared to solid beams and treatment systems of the past. Beyond the use of beams having a transverse symmetric annular profile are another kind of beam, which include those having a transverse asymmetric annular profile. Such laser beams having an asymmetric energy distribution can have other benefits as compared to those seen in the annular beams. As described herein, the MTZs created by annular beams can result in a ring-like shape of damaged tissue with a central region of spared tissue. However, in some instances, there is a chance that the central region of spared tissue at the center of the MTZ becomes nutritionally isolated and may not survive. In contrast, electromagnetic radiation (EMR) beams having an asymmetric energy distribution can create MTZs having an annular or semi-annular shape, such as crescents, semi-circles, ellipses, and elliptical rings, among others, which have an identifiable central region but that are not necessarily nutritionally isolated from a region outside the MTZ. Essentially, the asymmetric energy distribution can result in MTZs surrounded by a completely contiguous, undamaged tissue region, rather than a central region of spared tissue.

The present disclosure provides systems and devices that are able to consistently create beams having both symmetric and asymmetric energy distributions that result in the above-described MTZs, which provide numerous benefits for treatment procedures. A clinician operating such systems can switch between modes in which the outputted beam has a symmetric or an asymmetric distribution, depending upon treatment demands.

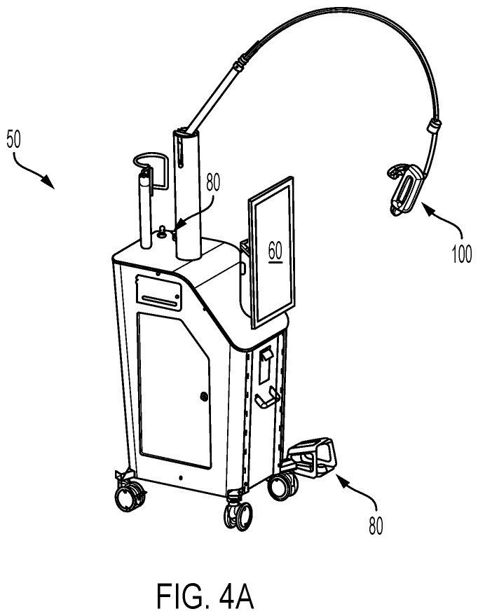

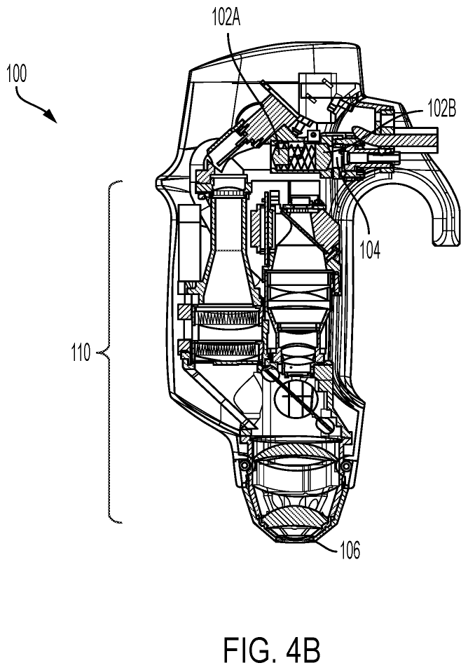

Referring now to FIGS. 4A and B, a treatment system 50 including a treatment device 100 is depicted. The treatment system 50 can be operated to treat tissue with symmetric and asymmetric beams as required for a give treatment. The treatment system 50 generally includes a display 60 for displaying information, a user interface 70 for receiving inputs for the treatment system 50, and a foot petal 80 for initiating a treatment procedure. The treatment device 100 can include an optical arrangement for use in the treatment device 100 according to some embodiments are shown. As explained above, the use of a treatment beam having a transverse annular profile is desirable to increase the effectiveness of a treatment procedure compared to a solid beam. The treatment device 100 can use a pair of axicons 102A, 102B (also referred to as a first axicon 102A and a second axicon 102B) to shape incident optical energy into a beam 100 having the desired transverse annular profile, which can be either symmetrical or asymmetrical. For example, the axicons 102A, 102B could be axicons from Thorlabs: AX2520-C. Optical energy 10 can be emitted by an electromagnetic radiation (EMR) source (not pictured) in electronic communication with the treatment device 100 toward a collimating lens 104 that acts to collimate the emitted optical energy 10 into a beam. The collimating lens 104, for example, can from Thorlabs: 354280-C. The collimated optical energy can then be shaped by the first and second axicons 102A, 102B into a beam having an annular profile. After being shaped by the first and second axicons 102A, 102B, the beam can be reflected by various reflective elements and through various optical elements before exiting the treatment device 10 through an optical window 106 toward a target tissue.

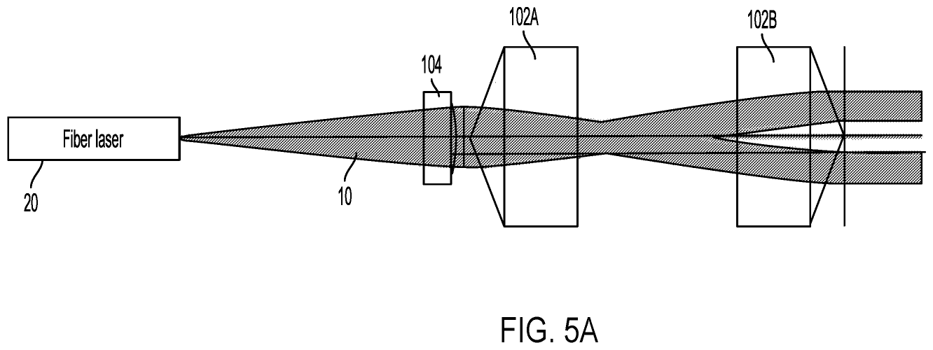

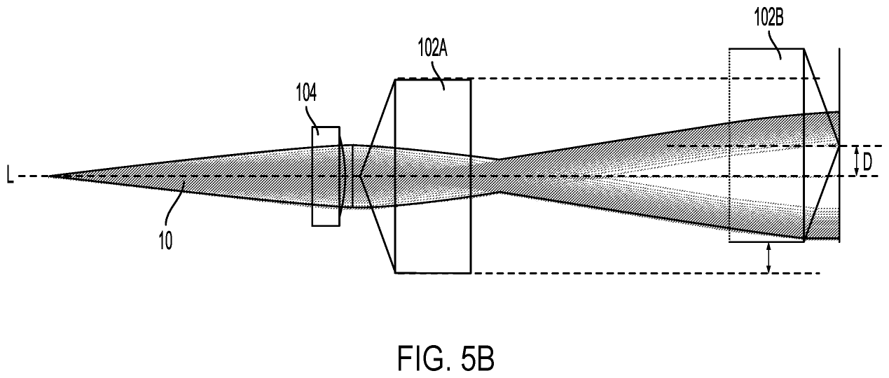

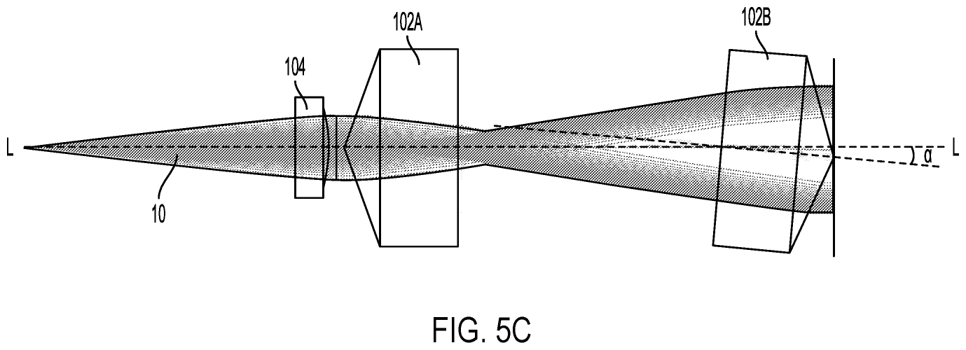

FIG. 5A depicts a simulation of the EMR source 20, the collimating lens 104, and the pair of axicons 102A, 102B in operation as they shape the beam 10. The simulation is performed with Ansys Optics Studio software using an IPG Photonics 1550 nm laser (ELR-20). A misalignment of the axicons 102A, 102B relative to each other and/or relative to the collimating lens 104 can ultimately result in the asymmetric energy distribution that is necessary to create the asymmetric beam. This misalignment can be realized in a number of ways, resulting in specific beam shapes and MTZ patterns like the shapes described above, including translation and/or rotation of one or both axicons 102A, 102B relative to each other. Even a slight misalignment can result in a large impact to the beam 10 downstream, which can impact a treatment procedure.

When the axicons are translated relative to each other, the beam 10 can be shaped to ultimately have an asymmetric profile, like the kind described herein. An example of translated axicons can be seen in FIG. 5B, which depicts a variation of the setup of FIG. 5A. While the depiction in FIG. 5B may not necessarily be to scale, the depiction of FIG. 5B gets across the general idea of a misalignment of optical elements by translation. In the variation of FIG. 5B, the collimating lens 104 and the first axicon 102A can be aligned about an optical axis L running through centers thereof. However, further downstream, the second axicon 102B can be translated by some displacement D (also called decenter) so that a center of the second axicon 102B is no longer aligned along the optical axis L. This displacement D may be slight—only a few fractions of a millimeter or more. For example, in one aspect, the diameter of the first and second axicons 102A, 102B can be about 12.7 mm, and the displacement D can be about 0.3 mm.

When the axicons are rotated relative to one another, the beam 10 can also be shaped to have an asymmetric profile. An example of axicons rotated relative to each other can be seen in FIG. 5C, which depicts another variation of the setup of FIG. 5A, not necessarily drawn to scale. In the depiction, similar to the variation of FIGS. 5A and 5B, both the collimating lens 104 and the first axicon 102A are aligned with the optical axis L. However, the second axicon 102B is rotated relative to the first axicon 102A and the optical axis L by some angle α, such that a leading face of the second axicon 102B is centered on the optical axis L while a trailing face of the second axicon 102B is not centered on the optical axis L. This rotation by the angle α also produces a beam 10 having an asymmetric profile as described herein.

In a comparable treatment system in which the axicons 102A, 102B were totally aligned, the beam 10 would be shaped to have a transverse annular profile centered on an optical axis of an optical window, which contacts the target tissue 50 during a treatment procedure. However, misaligned axicons 102A, 102B result in a beam 10 that is shifted so that it is no longer centered on an optical axis A of the optical window 106. The shaped beam 10 can be reflected and directed by one or more optical elements 110 in order to be directed and focused onto the target tissue 50.

While examples of an asymmetric beam resulting from misaligned axicons (either relative to themselves or to other elements, like the collimating lens 104) are provided, misalignment of any of the one or more optical elements 110 in the treatment device 100 can result in asymmetric beams of numerous shapes and sizes. For example, misalignment can occur relative to optical elements, the optical axis L, etc., and can involve the translation and/or rotation of one or more optical elements.

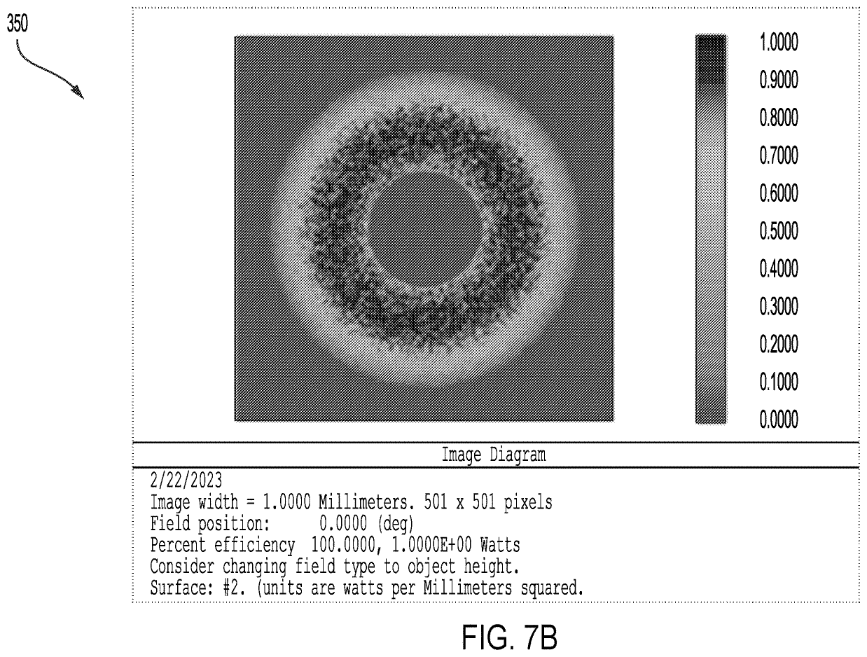

As mentioned above, a treatment laser beam creates MTZs in skin tissue, and the shape of the MTZs can depend upon the shape of the beam used to create the MTZs. FIGS. 7-10E depict the specific energy distribution of variously-shaped beams, as well as resulting MTZs, in greater detail.

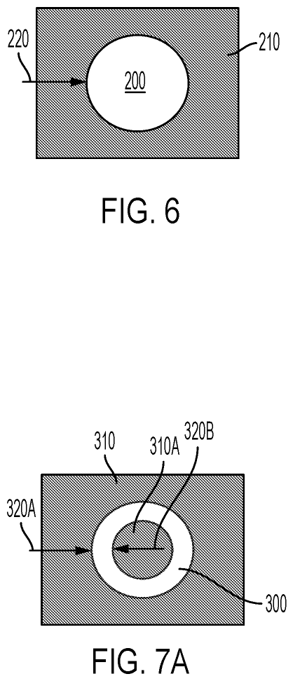

When a treatment laser beam is not shaped, an MTZ 200 results that is essentially circular, which can be seen in FIG. 6. After irradiation by such a beam, a healing process of the irradiated target tissue 210 occurs from the outside edge of the MTZ 200 in an inward direction, as indicated by the healing direction 220.

When a treatment laser beam is shaped to result into a beam having a symmetrical transverse annular energy profile an MTZ 300 results that is annular in shape, which can be seen in FIGS. 7A and 7B. FIG. 7A depicts a simplified view of a target tissue 310 upon which the annular MTZ 300 is created, while FIG. 7B depicts a plot 350 of the concentration of energy delivered to the target tissue 310 in an ideal beam to create the annular MTZ 300. In both FIG. 7A and FIG. 7B, the ring-like structure of the MTZ 300 is plainly depicted. After irradiation by such a shaped beam, a healing process of the irradiated target tissue 310 can occur from the outside edge of the MTZ 300 in an inward direction as indicated by the healing direction 320A, similar to the MTZ 200 of FIG. 6. However, the healing process can also occur from the inside edge of the MTZ 300 in an outward direction as indicated by the healing direction 320B. In some instances, healing only occurs in the healing direction 320A, such as when the target tissue 310A located within the MTZ 300 becomes isolated from other viable tissue and dies, as described above.

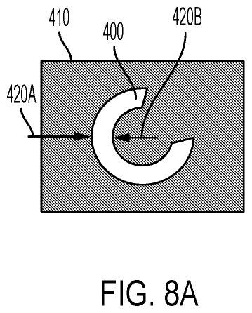

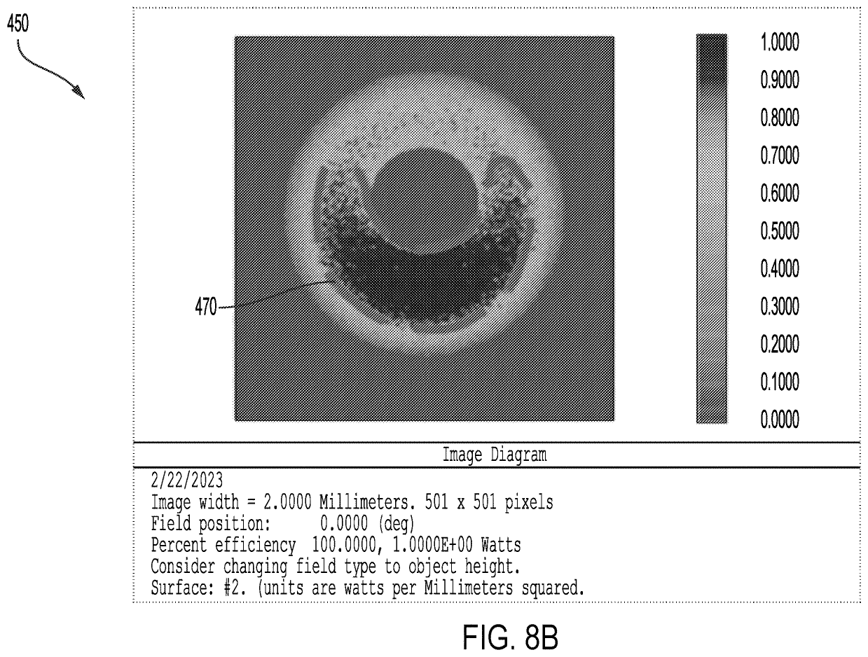

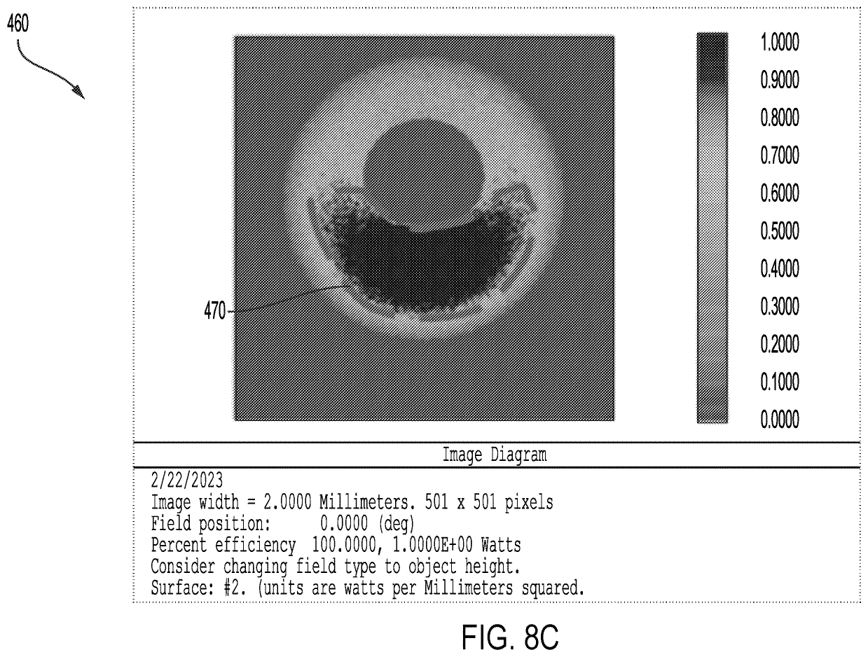

When a treatment laser beam is shaped to have an asymmetric beam shape, an MTZ 400 can result that has a partially-annular shape, which can be seen in FIGS. 8A-8C. Such shapes can include crescents, lunes, semi-annuli, and more, as well as shapes that are substantially like those listed geometric shapes, which can result as a consequence of the physiological features of the target tissue 410 itself. FIG. 8A depicts a simplified view of an MTZ 400 created on target tissue 410 in which the semi-annular appearance of the MTZ 400 is clearly defined. In contrast, FIGS. 8B and 8C depict plots 450, 460 of examples of the concentration of energy delivered to the target tissue 410, in which the plots 450, 460 depict a more rounded shape. All of the shapes depicted in FIGS. 8A-8C can result from a misalignment of various elements in the treatment device 10, including a misalignment of the axicons 102A, 102B, although these shapes are illustrative only, and are not meant to be limiting. For example, the plot 450 of FIG. 8B can result when the axicons 102A, 102B are decentered by about 0.3 mm, and the plot 460 of FIG. 8C can result when the axicons 102A, 102B are decentered by about 0.6 mm.

After irradiation by such a shaped beam, a healing process of the irradiated target tissue 410 can occur. An examination of the plots 450, 460 of FIGS. 8B and 8C reveals that the distribution of energy is essentially annular, with a clear delineation between outside untreated tissue, partially annular treated tissue, and inside untreated tissue. However, certain treated tissue does not receive enough optical energy and the MTZ 400 does not form in some areas. Therefore, during the healing process, healing can be described as occurring from the outside edge of the MTZ 400 in an inward direction as indicated by the healing direction 420 because the MTZ 400 effectively does not contain an isolated island of untreated tissue, such that healing can be said to occur from an inside area in an outward direction, like can be said of the MTZ 300 depicted in FIG. 7A. As a result, the risk of the isolated island of tissue dying, which can occur when using a beam having an annular profile as described above, virtually does not exist. In fact, both FIGS. 8B and 8C include a dashed line 470 that delineates a region of a higher concentration of optical energy, which can correspond to an area of expected tissue coagulation, resulting in the creation of the MTZ 400, in the target tissue 410.

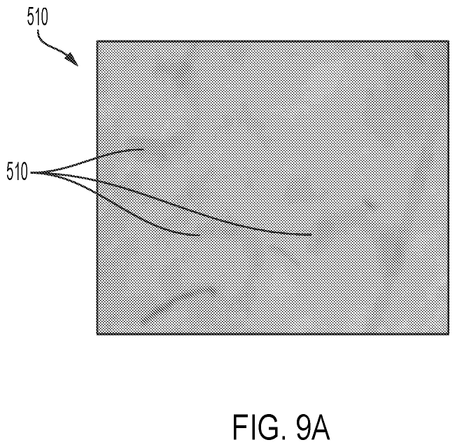

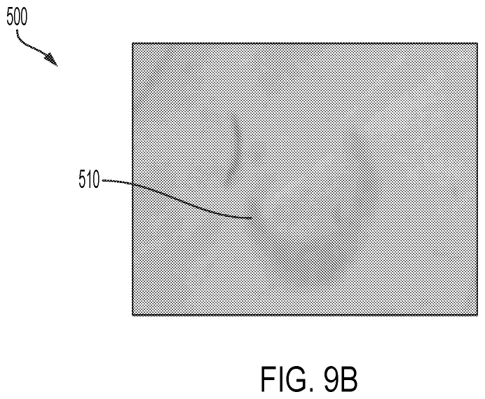

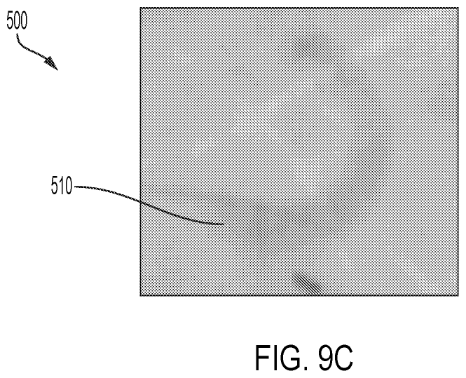

FIGS. 9A-9C depict tissue 500 that was treated by a beam having an asymmetric energy distribution. The tissue depicted is epidermal tissue 500 after receiving micro-thermal injuries from a 1550 nm laser. The laser creates what are known as MENDs (micro epidermal necrotic debris), which are brown regions of pigment. MENDs can appear in about 3 days after treatment and can clear in about 10 days after treatment, and the shape and location of MENDs is directly related to the creation of MTZs by a treatment beam during a treatment operation. As seen in FIGS. 9A-9C, when treated with a beam having an asymmetric energy profile like the kinds described herein, the MENDs 510 created by such a beam takes on a partially annular, cresecent-like shape, which lines up with the shape of the beam used to create them in the first place, similar to a beam having the distribution featured in FIGS. 9B and 9C. The images were captured with a commercial dermo-scope (DermLite Foto II Pro lens attached to a Nikon D500 camera).

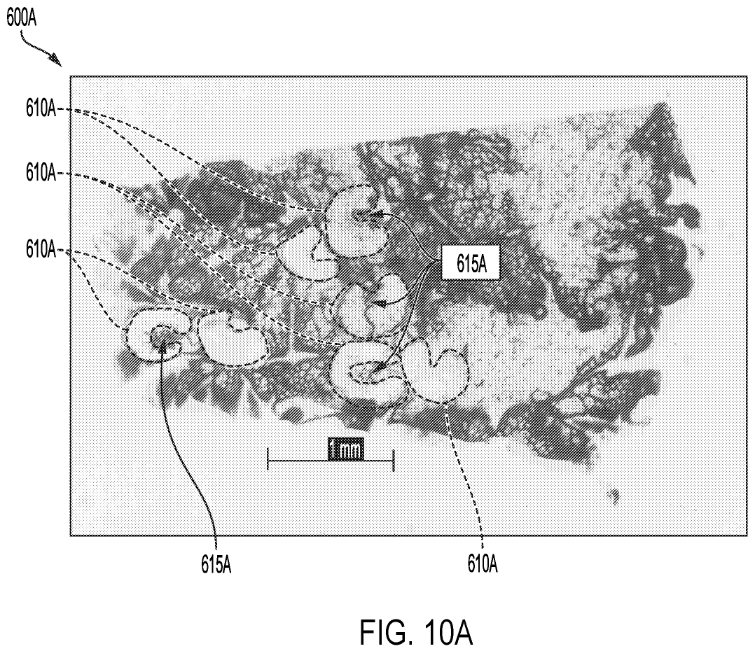

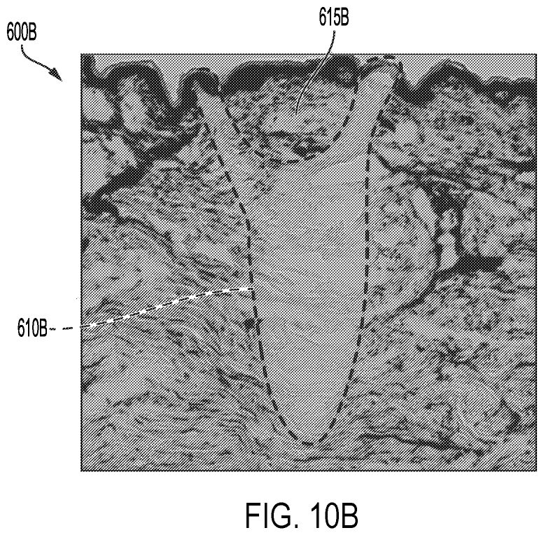

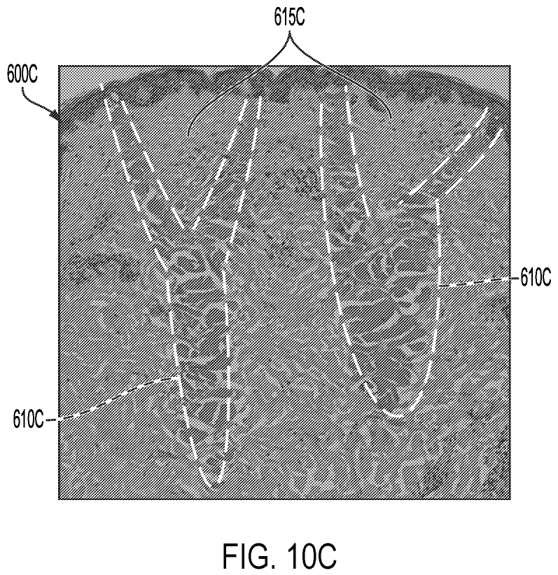

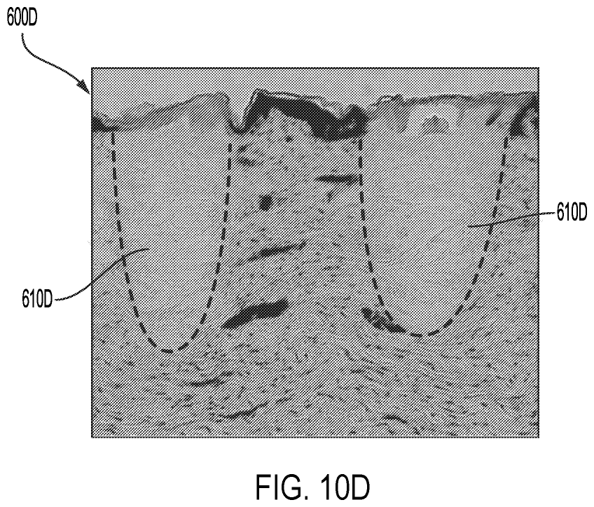

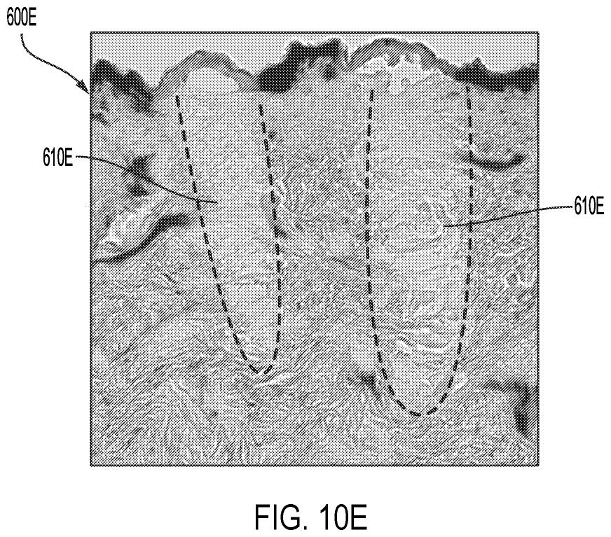

Effects of a beam having an asymmetric energy distribution can also be seen in FIGS. 10A-10E, which depicts histology of an en-face section of tissue 600A with NBTC staining taken immediately after treatment with a 1550 nm laser. The laser was focused to a depth of 1200 m, and energy was delivered in pulses at a rate of about 150 mJ per pulse. Lesions 610A having a partially annular or crescent-like shape can be seen in the tissue 600A, with boundaries of the lesions 610A marked in dashed lines to better highlight the lesions. Each lesion can also include a central island of viable tissue 615A that is preserved as a result of the shape of the laser. The shape of the non-viable areas of tissue closely matches the areas of highest intensity, which would be similar to the distributions seen in FIGS. 8B and 5C. Viable tissue appears blue and treated (or coagulated) tissue appears white due to lack of staining.

FIGS. 10B-10E depict additional views of cross-sections of tissue 600B-600E treated with an asymmetric beam. In FIGS. 10B and 10C, for example, lesions 610B, 610C damaged by a treatment beam are depicted as taking the form of a so-called “martini glass” shape. From the cross-section perspective, the spared tissue 615B, 615C can be readily seen. Each lesion 610B, 610C can also be seen extending deep into tissue and forming the “stem” of the martini glass. This extension can be due to scattering that light naturally experiences within tissue itself, which causes the overall lesion 610B, 610C to take on a shape that is different than an ideal cone as the light (and the energy it carries) “bleeds” outward due to said scattering to expand the bounds of the lesion 610A, 610B.

Of course, given the nature of the asymmetric beam as described herein, although the spared tissue 615B, 615C appears to be totally isolated, this is not necessarily the case. When viewed in three dimensions, the congruity of healthy tissue at each lesion will be apparent. For example, with reference to FIG. 10A, each of the lesions 610A would extend into the tissue along a direction that is normal to the image. If a cross-section of the lesions 610A were examined, the lesions 610A may appear differently depending upon where exactly the cross-section is taken. For example, as shown in FIGS. 10B and 10C, the lesions 610B, 610C appear to include isolated spared tissue regions 615B, 610C. However, in other examples, FIGS. 10D and 10E show cross-sections of lesions 610D, 610E made with a similar asymmetric beam as those described with respect to 10A-10C, however the lesions 610D, 610E do not include clearly defined spared tissue regions of any kind.

Ultimately, the size and shape of the lesion may depend upon numerous factors, including factors related to the treatment beam, the tissue, and/or other factors alone or in combination.

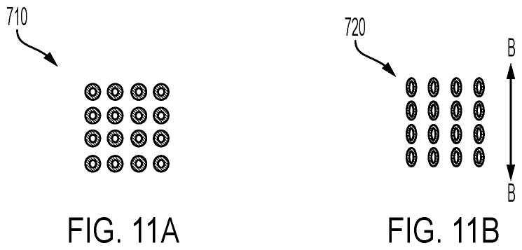

As mentioned above, misalignment of the one or more optical components relative to each other can result in a treatment beam having different shapes and being able to create MTZs of varying size and shape. One such beam geometry is an elongate or elliptical ring beam geometry, which can be similar to an ideal annular beam geometry described above, but stretched about a major axis thereof. Patterns created by such annular and elliptical ring beam geometries can be seen in FIGS. 11A and 11B. When treatment with an annular beam is performed, as seen in the array 710 of FIG. 11A, there is no preferential direction of the skin response. That is, when the skin is exposed to an annular beam and the skin coagulates in response to the received dosage of radiation, it tightens relative to its pre-radiation form. However, because annular beams are radially symmetrical, tightening and shrinking of the skin due to this coagulation would be the same about any radial direction. Some treatment applications of laser energy, such as a face-lift, require a pull (controlled tightening or shrinkage of the skin) in a specific direction. In order to achieve this pull in a specific direction, a beam having a geometry like the kind used to create the array 720 featured in FIG. 11B could be employed, which could promote skin tightening about a major axis B-B of the elliptical rings. In this way, the ring geometry could be harnessed to improve the efficacy of treatments that demand such directed tightening. While the specific shape of the beam is not critical, two features do matter: 1) the beam should not be radially symmetrical in order to encourage tightening in a specific direction; and 2) there should be an area of spared tissue in the middle of the MTZs created by a beam for the benefits described herein.

The subject matter described herein operated to create treatment EMR beams of varying parameters, including varying wavelength, average power, pulse duration, and more. For example, an operating wavelength can range from between about 1000 nm and 4000 nm, generally, between about 1200 and 3400 nm, and between about 1300 and 1900 nm. For example, the average power can be between about 10 W and 50 W, between about 14 W and 30 W, and between about 15 W and 25 W. For example, the pulse duration can be between about 0.5 ms and 10 ms, and between about 0.6 ms and 5 ms.

In an exemplary operation involving the system 50 and the treatment device 100, an operator enter certain patient information, either manually or through a pre-existing profile, via the display 60, user interface 70, or a remote device (not shown) such as a remote control, tablet, etc. This information can include, for example, skin type and/or any complications of the patient. The operator can select treatment areas for the patient and desired treatment zones/depths. Additionally, an operator can select whether certain portions of the patient are to be treated with a symmetric or asymmetric beam. Depending upon the beam selected, the treatment device 100 can adjust its internal components to align with the requirements described herein, such as through the use of various internal motors or galvanometers or the like. For example, the axicons 102A, 102B can be misaligned through translation and/or rotation as described above if an asymmetric treatment beam is desired.

Once the information is inputted into the system 50, a pre-scan can begin. The patient can be positioned as needed and a procedure gel can be applied to the treatment area. The procedure gel can assist with protecting skin from excessive thermal energy deposition as well as to assist in gliding the device 100 over the patient. Moreover, the gel can be designed so as not to disrupt the beam in any way as to compromise the procedure, and further, the gel can be designed to aid in the transmission of a treatment beam. Once positioned, the operator can select a scan zone on via the treatment system 50 via any number of standard inputs on the user interface 60, display 70, or the like (e.g., a touchscreen, keyboard, mouse, etc.).

To begin treatment, the operator can actuate a “ready” input and wait for the device 100 to cool. As described herein, the device 100 can include a cooling unit at a tip thereof, which can assist in patient comfort. The treatment beam can pass through the cooling unit without being disrupted. Once ready, the operator can actuate the footswitch 80 and move the device 100 across the patient's skin while maintaining contact between the tip of the device 100 and the tissue for the duration of the treatment. Multiple passes can be completed over the treatment area until the desired final density of treatment energy has been deposited on the treatment area. The system 50 can inform the operator when this takes place via any suitable method, such as an audible or visual alert. When treatment is complete, the operator can release the footswitch 80, remove the device 100 from the patient's skin, and return the system 50 to a standby mode.

The subject matter described herein can be implemented in digital electronic circuitry, or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processor of any kind of digital computer. Generally, a processor will receive instructions and data from a read only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of non-volatile memory, including by way of example semiconductor memory devices, (e.g., EPROM, EEPROM, and flash memory devices); magnetic disks, (e.g., internal hard disks or removable disks); magneto optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The techniques described herein can be implemented using one or more modules. As used herein, the term “module” refers to computing software, firmware, hardware, and/or various combinations thereof. At a minimum, however, modules are not to be interpreted as software that is not implemented on hardware, firmware, or recorded on a non-transitory processor readable recordable storage medium (i.e., modules are not software per se). Indeed “module” is to be interpreted to always include at least some physical, non-transitory hardware such as a part of a processor or computer. Two different modules can share the same physical hardware (e.g., two different modules can use the same processor and network interface). The modules described herein can be combined, integrated, separated, and/or duplicated to support various applications. Also, a function described herein as being performed at a particular module can be performed at one or more other modules and/or by one or more other devices instead of or in addition to the function performed at the particular module. Further, the modules can be implemented across multiple devices and/or other components local or remote to one another. Additionally, the modules can be moved from one device and added to another device, and/or can be included in both devices.

The subject matter described herein can be implemented in a computing system that includes a back end component (e.g., a data server), a middleware component (e.g., an application server), or a front end component (e.g., a client computer having a graphical user interface or a web browser through which a user can interact with an implementation of the subject matter described herein), or any combination of such back end, middleware, and front end components. The components of the system can be interconnected by any form or medium of digital data communication, e.g., a communication network. Examples of communication networks include a local area network (“LAN”) and a wide area network (“WAN”), e.g., the Internet.

Approximating language, as used herein throughout the specification and claims, may be applied to modify any quantitative representation that could permissibly vary without resulting in a change in the basic function to which it is related. “Approximately,” “substantially,” or “about” can include numbers that fall within a range of 1%, or in some embodiments within a range of 5% of a number, or in some embodiments within a range of 10% of a number in either direction (greater than or less than the number) unless otherwise stated or otherwise evident from the context (except where such number would impermissibly exceed 100% of a possible value). Accordingly, a value modified by a term or terms, such as “about,” “approximately,” or “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or all of the group members are present in, employed in, or otherwise relevant to a given product or process. Furthermore, it is to be understood that the disclosed embodiments provide all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the disclosed embodiments where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the disclosed embodiments, or aspects of the disclosed embodiments, is/are referred to as comprising particular elements, features, etc., certain embodiments of the disclosure or aspects of the disclosure consist, or consist essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification. For example, any one or more active agents, additives, ingredients, optional agents, types of organism, disorders, subjects, or combinations thereof, can be excluded.

Where ranges are given herein, embodiments of the disclosure include embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise. It is also understood that where a series of numerical values is stated herein, the disclosure includes embodiments that relate analogously to any intervening value or range defined by any two values in the series, and that the lowest value may be taken as a minimum and the greatest value may be taken as a maximum. Numerical values, as used herein, include values expressed as percentages.

Although a few variations have been described in detail above, other modifications or additions are possible.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A system, comprising:
an electromagnetic radiation (EMR) source configured to generate a treatment EMR beam;
a plurality of optical elements configured to shape and focus the treatment EMR beam to a focal point within a tissue, the plurality of optical elements including a first axicon and a second axicon;
a window assembly located down-beam from the plurality of optical elements and configured to contact the tissue, the window assembly being configured to transmit the treatment EMIR beam; and
a controller configured to control the EMR source to generate the treatment EMR beam during a treatment procedure,
wherein the first axicon and the second axicon are configured to shape the treatment EMR beam such that the shaped EMIR beam has a transverse asymmetric energy profile.

2. The system of claim 1, wherein the transverse asymmetric energy profile is at least partially annular.

3. The system of claim 2, wherein the transverse asymmetric energy profile is a crescent.

4. The system of claim 1, wherein the transverse asymmetric energy profile is a substantially elongate ring.

5. The system of claim 1, wherein the first axicon and the second axicon are misaligned relative to each other.

6. The system of claim 5, wherein the first axicon is translated relative to the second axicon.

7. The system of claim 5, wherein the first axicon is rotated relative to the second axicon.

8. A method, comprising:
contacting a tissue with a window assembly of a treatment device;
generating a treatment electromagnetic radiation (EMR) beam with an EMR source in electronic communication with the treatment device; and
shaping and focusing the treatment EMR beam toward the tissue with a plurality of optical elements to create one or more treatment zones on the tissue, the plurality of optical elements shaping the beam such that the shaped EMR beam has a transverse asymmetric energy profile.

9. The method of claim 8, wherein the plurality of optical elements include a first axicon and a second axicon being misaligned relative to an optical axis therethrough.

10. The method of claim 9, wherein the first axicon is translated relative to the second axicon.

11. The method of claim 9, wherein the first axicon is rotated relative to the second axicon.

12. The method of claim 9, further comprising collimating the treatment EMR beam with a collimating lens.

13. The method of claim 12, wherein the first axicon and second axicon are misaligned relative to the collimating lens.

14. The method of claim 8, wherein the one or more treatment zones at least partially annular.

15. The method of claim 14, wherein the one or more treatment zones are crescent-shaped.

16. The method of claim 8, wherein the one or more treatment zones are a substantially elliptical ring.

17. The method of claim 16, wherein the one or more treatment zones are substantially elliptical rings having a major axis aligned with a direction of tightening.

18. The method of claim 8, further comprising pulsing the treatment EMR beam while moving the treatment device relative to the tissue to create the one or more treatment zones.
